(19) United States
(12) Patent Application Publication
(10) Pub. No.: US 2017/0071954 A1
(43) Pub. Date: Mar. 16, 2017

(54) PEDIATRIC CHOLIC ACID FORMULATION

(71) Applicant: LABORATOIRES CTRS, Boulogne-Billancourt (FR)

(72) Inventors: Bernard Deschamps, Champagne-Vigny (FR); Antoine Ferry, Paris (FR); Andreas Vogel, Sevres (FR)

(21) Appl. No.: 14/855,698

(22) Filed: Sep. 16, 2015

(51) Int. Cl. A61K 31/575; A61K 9/50

(52) U.S. Cl. CPC A61K 31/575; A61K 9/5026

(57) ABSTRACT

The present invention relates to a formulation comprising cholic acid, for oral administration, where the unpleasant taste of cholic acid is masked.

### FIELD OF INVENTION

The present invention relates to pediatric drug formulation. Especially, this invention relates to the manufacturing of granules comprising a core comprising a drug having an unpleasant taste, which may be cholic acid, and a taste-masking coating. These granules can be further formulated in a dosage form, including either sprinkle capsule, dispersible tablet, orodispersible tablet, or sachet.

### BACKGROUND OF INVENTION

Cholic acid is well known for the treatment of inborn errors in primary bile acid synthesis in infants, children and adolescents. The deficiency in these primary bile acids synthesis leads to hepatic failure or to progressive neurological impairment. In some territories, cholic acid is also authorized for the treatment of peroxisomal disorders manifesting as Zellweger spectrum disorders.

A major drawback in the therapeutic use of cholic acid is that cholic acid is very bitter and said taste cannot be adequately masked by the addition of sweeteners and/or flavoring agents. Therefore cholic acid formulations found in the commerce are in the form of capsules. As these capsules have to be swallowed, these formulations cannot be used in pediatric treatment or for treating patients having swallowing problems. For babies and children who cannot swallow capsules, the pediatric treatment currently used is to add the contents of the capsule to expressed breast milk, juice or fruit puree, exposing these patients to the unpleasant taste of cholic acid.

A technique already known to improve the palatability of a substance with an unpleasant taste consists in coating the active ingredient formulation with organic polymer. However, in the case of bile acids, the use of said technique is problematic because their bitter taste is particularly pungent and because their poor solubility in aqueous media, a polymeric coating might modify their dissolution profile and jeopardize their bioavailability.

Therefore, the development of cholic acid for oral administration without an unpleasant taste, maintaining its dissolution profile compared to the existing form Orphacol®, and which is suitable for administration in pediatric treatment or to patients having swallowing difficulties or swallowing disability, is highly needed.

### DETAILED DESCRIPTION

In a first aspect, the present invention relates to a formulation comprising cholic acid, for oral administration, where the unpleasant taste of cholic acid is masked. Especially, the formulation encompasses coated cholic acid granules. According to one embodiment, the formulation encompasses coated cholic acid granules comprising cholic acid and at least one binder. According to one embodiment, the formulation encompasses coated cholic acid granules comprising cholic acid and at least one excipient. Advantageously, the formulation encompasses coated cholic acid granules comprising cholic acid, at least one binder and at least one excipient.

Also, the invention relates to a granule comprising a core comprising cholic acid and a coating comprising at least one taste-masking compound. According to one embodiment, the core of said granule further comprises at least one binder and/or at least one excipient. According to one embodiment the core of said granule further comprises at least one binder, preferably selected from natural polymer, synthetic polymer or sugar. According to one embodiment the core of said granule further comprises at least one excipient preferably selected from sugars, polyols, calcium salts and polymers.

In one embodiment the formulation comprises at least one excipient and at least one granule comprising a core comprising cholic acid and a coating comprising at least one taste-masking compound. According to one embodiment, the at least one taste-masking compound is selected from starches, polyvinylpyrrolidones, gelatin, methylcellulose, hydroxypropyl methyl cellulose, microcrystalline cellulose, ethyl cellulose and methacrylic acid copolymer and combinations thereof. According to one embodiment, the at least one taste-masking compound is selected from Eudragit®-type polymers (Evonik Industries AG, Darmstadt, Germany), ethylcellulose, sodium carboxymethylcellulose (Na-CMC) cellulose acetate butyrate, polyvinylpyrrolidone, hydroxyl ethyl cellulose, ethyl cellulose, polyvinyl acetate, crospovidone, croscarmellose, polycarbophil, polyacrylic acid, cellulose ethers, cellulose ester, polyvinyl acetate, methyl acrylate, hydroxyl ethyl methacrylate, vinyl pyridine, and combinations thereof. According to one embodiment, the at least one taste-masking compound is preferably selected from Eudragit® E-type polymers, Eudragit® RS-type polymers or Eudragit® RL-type polymers, more preferably selected from Eudragit® E PO or Eudragit® RL 30 D. According to one embodiment, the at least one taste-masking compound is preferably selected from Basic Butylated Methacrylate Copolymer or Ammonio Methacrylate Copolymer, more preferably selected from Poly(butyl methacrylate-co-(2-dimethylaminoethyl) methacrylate-co-methyl methacrylate) (1:2:1) or Poly(ethyl acrylate-co-methyl methacrylate-co-trimethylammonioethyl methacrylate chloride) (1:2:0.2).

According to one embodiment the binder may be selected from natural polymer, synthetic polymer or sugar. According to one embodiment, the at least one binder is selected from starch, pregelatinized starch, gelatin, acacia, alginic acid, sodium alginate, polyvinylpyrrolidone (PVP), methylcellulose, hydroxylpropylcellulose (HPC), hypromellose (HPMC), sodium carboxymethylcellulose (Na-CMC), ethylcellulose, glucose, sucrose, sorbitol and combinations thereof. According to one embodiment, the binder is preferably polyvinylpyrrolidone (PVP) or hydroxylpropylcellulose (HPC).

According to one embodiment, the at least one excipient is selected from sugars, polyols, calcium salts and polymers. According to one embodiment, the other excipient or combinations of excipients are selected from sucrose, lactose, maltose, lactulose, trehalose, cellobiose, melibiose, isomaltose, glycerol, erythriol, xylitol, arabitol, ribitol, sorbitol, dulcitol, mannitol, volemitol, maltitol, isomaltitol, lactitol, calcium carbonate, dicalcium phosphate, tricalcium phosphate, microcrystalline cellulose and combinations thereof. According to one embodiment, the other excipient or combinations of excipients is a combination of microcrystalline cellulose and mannitol.

According to one embodiment the cholic acid granules are formulated in a pharmaceutical formulation, including either sprinkle capsule, dispersible tablet, orodispersible tablet, or sachet by mixing the granules with one or several excipients such as: diluent, disintegrant, lubricant.

According to one embodiment the diluent may be selected from sugars, polyols, calcium salts and polymers. According to one embodiment, the at least one excipient is selected from sucrose, lactose, maltose, lactulose, trehalose, cellobiose, melibiose, isomaltose, glycerol, erythriol, xylitol, arabitol, ribitol, sorbitol, dulcitol, mannitol, volemitol, maltitol, isomaltitol, lactitol, calcium carbonate, dicalcium phosphate, tricalcium phosphate, microcrystalline cellulose and combinations thereof. According to one embodiment, the diluent is preferably microcrystalline cellulose.

According to one embodiment the disintegrant may be selected from natural polymer or synthetic polymer. According to one embodiment, the disintegrant is selected from povidone, crospovidone, microcrystalline cellulose, sodium croscarmellose, methylcellulose, starch, pregelatinized starch, carboxymethyl starch, alginic acid, sodium alginate. According to one embodiment, the disintegrant is preferably crospovidone or sodium croscarmellose.

According to one embodiment the lubricant may be selected from hydrophobic or hydrophilic lubricants. According to one embodiment, the at least one lubricant is selected from stearic acid, magnesium stearate, sodium stearyl fumarate. According to one embodiment, the lubricant is preferably sodium stearyl fumarate or magnesium stearate.

In a second aspect, the present invention relates to a process for manufacturing the formulation, comprising at least one granule comprising a core comprising cholic acid and a coating comprising at least one taste-masking compound, comprising:
Granulating cholic acid;
Coating the said granules with at least one taste-masking compound; and
Mixing cholic acid with at least one excipient.

The above steps may be carried out in various ways.

According to a first embodiment, steps of the said process for the manufacturing of formulation comprising at least granule comprising a core comprising cholic acid and a coating comprising at least one taste-masking compound are performed in the following order:
a1. Granulating the said cholic acid;
b1. Coating the said granules with at least one taste-masking compound; and
c1. Mixing coated cholic acid granules with at least one excipient.

According to second embodiment, steps of the said process for the manufacturing of formulation comprising at least one granule comprising cholic acid and at least one taste-masking compound are performed in the following order:
a2. Mixing cholic acid with at least one excipient;
b2. Granulating the said cholic acid with at least one excipient;
c2. Coating the said granules with at least one taste-masking compound.

According to a third embodiment, steps of the said process for the manufacturing of formulation comprising at least granule comprising a core comprising cholic acid and at least one excipient and a coating comprising at least one taste-masking are performed in the following order:
a3. Mixing cholic acid with at least one excipient;
b3. Granulating the said cholic acid with at least one excipient;
c3. Coating the said granules with at least one taste-masking compound; and
d3. Mixing coated cholic acid granules with at least one excipient.

According to a fourth embodiment, steps of the said process for the manufacturing of formulation comprising at least one granule comprising a core comprising cholic acid and at least one excipient and a coating comprising at least one taste-masking compound to be tabletted or to be filled-in sprinkle capsule or sachets are performed in the following order:
a4. Mixing cholic acid with at least one excipient;
b4. Granulating the said cholic acid with at least one excipient;
c4. Coating the said granules with at least one taste-masking compound;
d4. Mixing coated cholic acid granules with at least one excipient; and
e4. Tabletting the final blend to obtain dispersible or orodispersible tablets or filling the final blend in sprinkle capsules or sachets.

The present invention relates also to a process for manufacturing at least one granule of cholic acid comprising
Mixing cholic acid with at least one excipient;
Granulating the said cholic acid with at least one excipient using a wet granulation and drying the obtained granules; and
Coating the said granules with at least one taste-masking compound.

According to one embodiment, granulation can be a dry granulation performed with a swaying granulator (e.g. High Efficiency Swaying Granulator manufactured by Changzhou Kewei Mechanical Manufacturing Co., Ltd) or a high shear mixer-granulator (e.g. Aeromatic-Fielder™ manufactured by GEA Pharma Systems), or a wet granulation performed with air in a fluidized bed dryer granulator (e.g. GPCG1 Top Spray manufactured by Glatt) or an impeller in a high shear granulator (e.g. VG25 manufactured by Glatt), or screws in a twin screw granulator (e.g. Pharma 24 TSG Twin Screw Granulator manufactured by Thermo Scientific).

According to one embodiment, the granulation is a wet granulation. The liquid solution used for the wet granulation can be aqueous based or solvent based. Typical liquids include water, ethanol, isopropanol and combinations thereof.

According to one embodiment, the wet granulation is performed with a high shear impeller or a fluidized bed dryer granulator.

According to one embodiment, the wet granulation is performed with a high shear impeller using an aqueous liquid solution.

According to one embodiment the liquid solution can include a binder. The binder is dissolved in water or solvent and added to the process.

According to one embodiment, the wet granules are discharged and dried in a fluidized bed dryer.

According to one embodiment, coating can be performed in a fluidized bed dryer granulator (e.g. GPCG1 Wurster manufactured by Glatt) using aqueous based or solvent based liquid solution; more preferably coating is performed in a fluidized bed dryer granulator using aqueous based liquid solution.

According to one embodiment, the cholic acid granules have a particle size distribution before coating with at least one taste-masking compound ranging from 30 μm to 710 μm, preferably from 30 μm to 355 μm, more preferably from 30 μm to 250 μm.

According to one embodiment, the particle size distribution of said cholic acid granules after coating ranging from 30 μm to 710 μm, preferably from 90 μm to 500 μm, more preferably from 125 μm to 355 μm.

According to one embodiment, the amount of cholic acid ranging from 1% w/w to 90% w/w, preferably from 5% w/w to 60% w/w, more preferably from 10% w/w to 40% w/w in weight to the total weight of cholic acid granules and excipient.

According to one embodiment, the amount of said taste-masking compound ranging from 1% w/w to 90% w/w, preferably from 5% w/w to 50% w/w, more preferably from 10% w/w to 40% w/w, in weight to the total weight of cholic acid granules and excipient.

According to one embodiment, the wet granulation step may include addition of a binder, preferably in an amount ranging from more than 0% w/w to 30% w/w, preferably from more than 0% w/w to 20% w/w, more preferably from 1% w/w to 10% w/w, in weight to the total weight of cholic acid granules and excipient.

According to one embodiment, the amount of binder, taste masking and excipient ranging from 15% w/w to 95% w/w, preferably from 40% w/w to 90% w/w, more preferably from 50% w/w to 80% w/w, in weight to the total weight of cholic acid granules and excipient.

According to one embodiment, the amount of said diluent ranging from 0% w/w to 90% w/w, preferably from 5% w/w to 70% w/w, more preferably from 10% w/w to 60% w/w, in weight to the total weight of cholic acid pharmaceutical formulation.

According to one embodiment, the amount of said disintegrant ranging from 0% w/w to 15% w/w, preferably from 2% w/w to 12% w/w, more preferably from 2% w/w to 10% w/w, in weight to the total weight of cholic acid pharmaceutical formulation.

According to one embodiment, the amount of said lubricant ranging from 0% w/w to 10% w/w, preferably from 0.1% w/w to 5% w/w, more preferably from 0.25% w/w to 2% w/w, in weight to the total weight of cholic acid pharmaceutical formulation.

In a third aspect, the present invention relates to a pharmaceutical formulation comprising at least one granule of cholic acid. The pharmaceutical formulation comprises at least one granule comprising cholic acid and at least one taste-masking compound. In one embodiment, the pharmaceutical formulation further comprises at least one binder and/or at least one excipient. Advantageously, the pharmaceutical composition comprises cholic acid, at least one taste-masking compound, at least one binder and at least one excipient.

In one embodiment the cholic acid granules are formulated in a pharmaceutical formulation, including either sprinkle capsule, dispersible tablet, orodispersible tablet, or sachet by mixing the granules with one or several excipients such as: diluent, disintegrant, lubricant.

In a fourth aspect, the present invention relates to a medicament comprising at least one granule as described above or the pharmaceutical composition as described above.

In one embodiment, the medicament is in an oral form, selected from the group consisting of sprinkle capsule, dispersible tablet, orodispersible tablet, or sachet.

In a fourth aspect, the present invention relates to a formulation comprising an effective amount of cholic acid for use in the treatment of a patient suffering from a deficiency in primary bile acid synthesis.

According to one embodiment, the patient is aged 1 month to 6 years.

According to one embodiment, the patient has swallowing difficulty or swallowing disability.

In a fifth aspect, the present invention relates to a unit dosage form comprising a container containing said coated cholic acid granules, optionally mixed with excipient granules.

In one embodiment, the unit dosage form comprises a container containing a formulation of the invention.

In one embodiment, the container is a sachet or a sprinkle capsule.

In a sixth aspect, the present invention relates to a method of treating a patient suffering from a deficiency in primary bile acid synthesis comprising administering a formulation comprising an effective amount of at least one granule comprising cholic acid and a coating comprising at least one taste-making compound.

Definitions

In the present invention, the following terms have the following meanings:
“Binder” refers to pharmaceutical glue dissolved in water or solvent and added to the wet granulation process. The binder forms a bond with the powders during the granulation process.
“Core” refers to the central or innermost part;
“Eudragit-type polymer” refers to poly(meth)acrylates for pharmaceutical applications described in Ph.Eur as Methacrylic Acid—Ethyl Acrylate Copolymer, Methacrylic Acid—Methyl Methacrylate Copolymer, Basic Butylated Methacrylate Copolymer, Ammonio Methacrylate Copolymer, Polyacrylate Dispersion;
“Excipient” refers to natural or synthetic substance formulated alongside the active ingredient of a medication included for the purpose of bulking up formulations that contain potent active ingredients (thus often referred to as “bulking agents,” “fillers,” or “diluents”), or to confer a therapeutic enhancement on the active ingredient in the final dosage form, such as facilitating drug absorption or solubility.
“Granulation” refers to the act or process in which primary powder particles are made to adhere to form larger, multiparticle entities called granules;
“Granule” refers to multiparticle entities;
“Taste-masking compound” refers to a compound used to mask the bitter and/or unpleasant taste of the active ingredient.

### EXAMPLES

The present invention is further illustrated by the following examples.

### Example 1

### Manufacturing of the Cholic Acid Pediatric Formulation with Granulation, Coating then Mixing with an Excipient

### Formulation 1

The following table presents the formulation of the cholic acid pediatric formulation.

| Chemical name ingredient | Dosage unit (mg) | Quantity % (w/w) |
|---|---|---|
| Cholic acid (CA) | 25.00 | 16.67% |
| PVP | 2.50 | 1.66% |
| Eudragit E PO | 5.50 | 3.67% |
| Maltitol Sweetpearl Roquette | 117.00 | 78.0% |
| Total | 150.00 | 100.0% |

Process of Manufacturing of the Pediatric Formulation

363.64 g of cholic acid were mixed with 36.36 g of polyvinylpyrrolidone K30 and then granulated by wet granulation using a high shear impeller. Water was used as granulation liquid. Cholic acid granules were then dried in a fluidized bed dryer (Glatt GPCG1). 400 g of cholic acid granules were then coated with an aqueous solution of Eudragit EPO in a Wurster fluidized bed dryer granulator to achieve a mass of 5.5 mg for 25 mg of cholic acid (corresponding to 80 g of dry Eudragit EP O). Spraying rate was of 6-7 g/min was applied and parameters were adjusted to ensure the product temperature was comprised between 25 and 30° C. Coated cholic acid granules were then dried and parameters were adjusted to ensure the temperature of the product to be not more than 40° C.

Finally the powder was mixed with qs 100% of maltitol Sweetpearl Roquette.

### Example 2

### Manufacturing of the Cholic Acid Pediatric Formulation with Granulation with an Excipient, then Coating

### Formulation 2

The following table presents the formulation of the cholic acid pediatric formulation.

| Chemical name ingredient | Dosage unit (mg) | Quantity % (w/w) |
|---|---|---|
| Cholic acid (CA) | 25.00 | 16.67% |
| Dicalcium phosphate | 87.50 | 58.33% |
| PVP | 11.25 | 7.50% |
| Eudragit E PO | 26.25 | 17.50% |
| Total | 150.00 | 100.0% |

Process of Manufacturing of the Pediatric Formulation

80.7 g of cholic acid were mixed with 36.3 g of polyvinylpyrrolidone K30 and 282 g of dicalcium phosphate and then granulated by wet granulation using a high shear impeller. Water was used as granulation liquid. Cholic acid granules were then dried in a fluidized bed dryer (Glatt GPCG1).

400 g of Cholic acid granules were then coated with an aqueous solution of Eudragit EPO in a Wurster fluidized bed dryer granulator to achieve a mass of 26.25 mg for 25 mg of Cholic acid (corresponding to about 84 g of dry Eudragit EP O).

Spraying rate was of 6-7 g/min was applied and parameters were adjusted to ensure the product temperature was comprised between 25 and 30° C. Coated cholic acid granules were then dried and parameters were adjusted to ensure the temperature of the product to be not more than 40° C.

### Example 3

### Manufacturing of the Cholic Acid Pediatric Formulation with Granulation with Excipients, then Coating and Tabletting

### Formulation 3

| Chemical name ingredient | Dosage unit (mg) | Quantity % (w/w) |
|---|---|---|
| Cholic acid (CA) | 25.00 | 16.67% |
| Mannitol | 10 | 6.67% |
| Microcrystalline cellulose | 10 | 6.67% |
| HPC | 1.40 | 0.93% |
| Eudragit E PO | 24.14 | 16.09% |
| Microcrystalline cellulose | 72.71 | 48.47% |
| Sodium croscarmellose | 6.00 | 4.00% |
| Magnesium stearate | 0.75 | 0.50% |
| Total | 150.00 | 100.00% |

Process of Manufacturing of the Pediatric Formulation

377.16 g of cholic acid was mixed with 21.12 g of HPC, 150.86 g of microcrystalline cellulose and 150.86 g of mannitol and then granulated by wet granulation using a high shear impeller. Water was used as granulation liquid. Cholic acid granules were then dried in a fluidized bed dryer (Retsch).

628.84 g of the prepared Cholic acid granules were then coated with an aqueous solution of Eudragit EPO in a Wurster fluidized bed dryer granulator to achieve a mass of 24.14 mg for 25 mg of Cholic acid (corresponding to 327.16 g of dry Eudragit EP O).

Spraying rate was of 8-10 g/min was applied and parameters were adjusted to ensure the product temperature was comprised between 25 and 35° C. Coated cholic acid granules were then dried and parameters were adjusted to ensure the temperature of the product to be not more than 40° C.

117.57 g of the coated cholic acid granules were mixed with 121.18 g of microcrystalline cellulose and 10.00 g of sodium croscarmellose and blended during 5 minutes at 2×22 rpm. 1.25 g of Magnesium stearate was then added and the mixture was blended during 2 minutes at 2×22 rpm. The final blend was then tabletted on a rotative press to obtain dispersible tablets.

### Example 4

### Bitterness test

### Formulation 4

### EthylCellulose

| Chemical name ingredient | Dosage unit (mg) | Quantity % (w/w) |
|---|---|---|
| Cholic acid (CA) | 25.00 | 81.7% |
| PVP | 2.17 | 7.1% |
| Ethylcellulose + Hypromellose | 3.43 | 11.2% |
| 2910 | | |
| Total | 30.60 | 100.0% |

### Formulation 5

### Eudragit RL30D+Carboxymethylcellulose Sodique (Na-CMC)

| Chemical name ingredient | Dosage unit (mg) | Quantity % (w/w) |
|---|---|---|
| Cholic acid (CA) | 25.00 | 81.94% |
| PVP | 2.17 | 7.13% |
| Eudragit RL 30D + Na-CMC + | 3.33 | 10.93% |
| Polysorbate 80 + Talc | | |
| Total | 30.50 | 100.0% |

### Formulation 6

### Eudragit E PO

| Chemical name ingredient | Dosage unit (mg) | Quantity % (w/w) |
|---|---|---|
| Cholic acid (CA) | 25.00 | 81.94% |
| PVP | 2.17 | 7.13% |
| Eudragit E PO | 3.33 | 10.93% |
| Total | 30.50 | 100.0% |

Process of Manufacturing of the Pediatric Formulation

Cholic acid was mixed with polyvinylpyrrolidone K30 granulated by wet granulation using a Top Spray fluidized bed dryer granulator (Glatt GPCG1). Water was used as granulation liquid. Spraying rate was of 10-12 g/min was applied and parameters were adjusted to ensure the product temperature was comprised between 25 and 35° C. Coated cholic acid granules were then dried and parameters were adjusted to ensure the temperature of the product to be not more than 40° C.

Cholic acid granules were then coated with a taste-masking compound in a Wurster fluidized bed dryer granulator. Spraying rate of 6-7 g/min was applied and parameters were adjusted to ensure the product temperature was comprised between 25 and 45° C. Coated cholic acid granules were then dried and parameters were adjusted to ensure the temperature of the product to be not more than 45° C.

Results

The formulations 1-2-3-4-5-6 were then swallowed as it is, or/and in suspension in water to evaluate their bitterness. The rate of emergence of the bitterness and its intensity were then evaluated for each formulation by a panel of 5 persons. A score between 0 and ++ was assigned. ++ means that the formulation is highly bitter and 0 means there is no bitterness.

| | | Rate of emergence of the bitterness | |
|---|---|---|---|
| Formulation | Intensity | Dry | Suspension in water |
| Formulation 1 | 0 | − | No bitter taste |
| Formulation 2 | 0 | − | No bitter taste |
| Formulation 3 | 0 | − | No bitter taste |
| Formulation 4 | ++ | 0-10 seconds | ~10 seconds |
| Formulation 5 | ++ | 0-10 seconds | ~10 seconds |
| Formulation 6 | + | 0-10 seconds | ~10 seconds |

1. Granule comprising a core comprising cholic acid and a coating comprising at least one taste-masking compound.

2. Granule according to claim 1, wherein the core of said granule further comprises at least one binder.

3. Granule according to claim 1, wherein the core of said granule further comprises at least one excipient.

4. Granule according to claim 1, wherein the at least one taste-masking compound is selected from starches, polyvinylpyrrolidones, gelatin, methylcellulose, hydroxypropyl methyl cellulose, microcrystalline cellulose, ethyl cellulose and methacrylic acid copolymer and combinations thereof.

5. Formulation comprising at least one granule comprising cholic acid and at least one taste-masking compound according to claim 1, wherein the formulation further comprises at least one excipient.

6. Formulation according to claim 5, wherein the amount of cholic acid ranging from 1% w/w to 90% w/w in weight to the total weight of cholic acid granules and excipient.

7. Formulation according to claim 5, wherein the amount of said taste-masking compound ranging from 1% w/w to 90% w/w in weight to the total weight of cholic acid granules and excipient.

8. Method of treating a patient suffering from a deficiency in primary bile acid synthesis comprising administering a formulation according to claim 5 comprising an effective amount of at least one granule comprising cholic acid and a coating comprising at least one taste-making compound.
